(19) United States
(12) Patent Application Publication
(10) Pub. No.: US 2020/0008895 A1
(43) Pub. Date: Jan. 9, 2020

(54) INTRACORPOREAL ASSEMBLED DEVICE FOR MINIMALLY INVASIVE SURGERY

(71) Applicant: SHOW CHWAN MEMORIAL HOSPITAL, Changhua City (TW)

(72) Inventors: Chun-Min SU, Lukang Township (TW); Siang-Cyun WANG, Lukang Township (TW)

(21) Appl. No.: 16/506,210

(22) Filed: Jul. 9, 2019

Related U.S. Application Data
(60) Provisional application No. 62/695,200, filed on Jul. 9, 2018.

(51) Int. Cl. A61B 34/00; A61B 17/29; A61B 17/04

(52) U.S. Cl. CPC A61B 34/70; A61B 2017/00292; A61B 17/0469; A61B 17/2909

(57) ABSTRACT

An intracorporeal assembled device for minimally invasive surgery is provided. The intracorporeal assembled device for minimally invasive surgery is applied to a laparoscopic surgery and includes a hand piece and a surgical piece. The hand piece has a handle portion, the handle portion has a pipe portion, the pipe portion has a first diameter, and a first connection portion is disposed to one end of the pipe portion. The surgical piece has a second diameter, a second connection portion corresponding to the first connection portion is disposed to one end of the surgical piece, and a workpiece is disposed to one other end of the surgical piece. The first diameter is smaller than the second diameter, and the surgical piece is connected to the pipe portion in a detachable manner via the first connection portion and the second connection portion.

### CROSS-REFERENCE TO RELATED APPLICATION

This application claims the benefits of U.S. provisional application No. 62/695,200, filed on Jul. 9, 2018, the content of which is hereby incorporated by reference in their entirety for all purposes.

### BACKGROUND OF THE INVENTION

### Field of the Invention

The present invention relates to surgical devices and instruments. Particularly, the present invention relates to an intracorporeal assembled device for minimally invasive surgery, which is detachable and applicable to an abdomen minimally invasive surgery.

### Description of the Related Art

In a minimally invasive surgery, a thin and long surgical instrument is usually used by the doctor to perform the surgery. In operation, one end of the surgical instrument is held by the doctor while the other end of the surgical instrument passes through a small wound opening to probe inside the body of the patient.

Therefore, the surgical instrument is the only portion contacting the lesion tissue of the patient, and also the only tool for directly conducting the surgery. The motion of the handheld portion of the surgical instrument maps to the motion of the distal end contacting the lesion tissue. During the surgery, in order to achieve different operations such as holding, stitching and knotting, the doctor needs to hold the surgical instrument and make big moves, which limits the flexibility of the end contacting the lesion tissue and increases the difficulty of the surgery. The main problems with existing products are that degree of operation freedom at the end of the instrument is limited, the operation space is small, and the operation power is insufficient. The operation flexibility of the instrument is hence low and many complicated operation actions cannot be performed. The other problem is the port size. The port size is directly related to the postoperative pain. The trend of minimally invasive surgery is toward to three aspects which are reduced port surgery (e.g. from multiple ports to single port surgery), natural orifice transluminal endoscopic surgery (NOTES), and reduced port size surgery (e.g. trocar size from 12 mm, 11 mm, 5 mm, 3.5 mm to 3 mm in diameter), also known as needle surgery. The drawbacks of needle surgery are easily broken instruments, limited function of instruments (e.g. difficult to clip vessels) due to small caliber of instruments. The present invention discloses the solution to these drawbacks.

Consequently, the inventor of the present invention has studied for many years and designed an intracorporeal assembled device for minimally invasive surgery to redeem the lack of the prior art and enhance the implementation and utilization of the industry.

### SUMMARY OF THE INVENTION

In view of the problems of the prior art, the present invention aims to provide an intracorporeal assembled device for minimally invasive surgery to redeem the lack of the prior art and enhance the applicability of the needle operation in the minimally invasive surgery.

According to an object of the present invention, an intracorporeal assembled device for minimally invasive surgery is provided. The intracorporeal assembled device for minimally invasive surgery is applied to a laparoscopic surgery and includes a hand piece and a surgical piece. The hand piece has a handle portion, the handle portion has a pipe portion, the pipe portion has a first diameter, and a first connection portion is disposed to one end of the pipe portion. The surgical piece has a second diameter, a second connection portion corresponding to the first connection portion is disposed to one end of the surgical piece, and a workpiece is disposed to one other end of the surgical piece. The first diameter is smaller than the second diameter, and the surgical piece is connected to the pipe portion in a detachable manner via the first connection portion and the second connection portion.

Preferably, the first diameter may be smaller than or equal to 3.5 mm.

Preferably, the second diameter may be greater than 3.5 mm.

Preferably, the first connection portion and the second connection portion may be magnetic members of different magnetic poles.

Preferably, the first connection portion and the second connection portion may be members combining with each other as a buckle set.

Preferably, the hand piece may include a wire, the wire connects to the handle portion to be controlled by the handle portion, and the wire passes through the pipe portion and connects to the surgical piece.

Preferably, the workpiece may be a camera.

Preferably, the hand piece may be a straight handle or a horizontal handle.

Preferably, the handle portion may further include a fixed handle portion and a substitutive handle portion, the pipe portion is disposed to the fixed handle portion, and the substitutive handle portion connects to the fixed handle portion in a detachable manner.

Preferably, the workpiece may be a clamp, a stitching tool, a knotting tool or an energy device.

Preferably, the first connection portion and the second connection portion may be a mortise-and-tenon structure.

The technical features of the present invention will be described in detail in specific embodiments with reference to the accompanying drawings such that a person skilled in the art may easily appreciate the purpose, technical features and advantages of the present invention.

### BRIEF DESCRIPTION OF THE DRAWINGS

FIG. 1 depicts the first structure of the intracorporeal assembled device for minimally invasive surgery of the present invention.

FIG. 2 depicts the second structure of the intracorporeal assembled device for minimally invasive surgery of the present invention.

FIG. 3 depicts the structure of the hand piece of the intracorporeal assembled device for minimally invasive surgery of the present invention.

FIG. 4 depicts the structure of the hand piece and surgical piece of the intracorporeal assembled device for minimally invasive surgery of the present invention.

FIG. 5 depicts the example of implementation of the intracorporeal assembled device for minimally invasive surgery of the present invention.

### DETAILED DESCRIPTION OF THE PREFERRED EMBODIMENTS

The advantages, features and technical methods of this invention will be described in detail in order to be understood easier. Moreover, the present invention may be realized in different form and should not be limited to the embodiments described here. On the contrary, the provided embodiments make the disclosure more clear and define the scope of this invention entirely and completely. Further, the present invention is only defined according to the attached claims.

Embodiments of the intracorporeal assembled device for minimally invasive surgery according to the present invention will be described below with reference to the related drawings. For ease of understanding, the same components in the following embodiments are denoted by the same reference numerals.

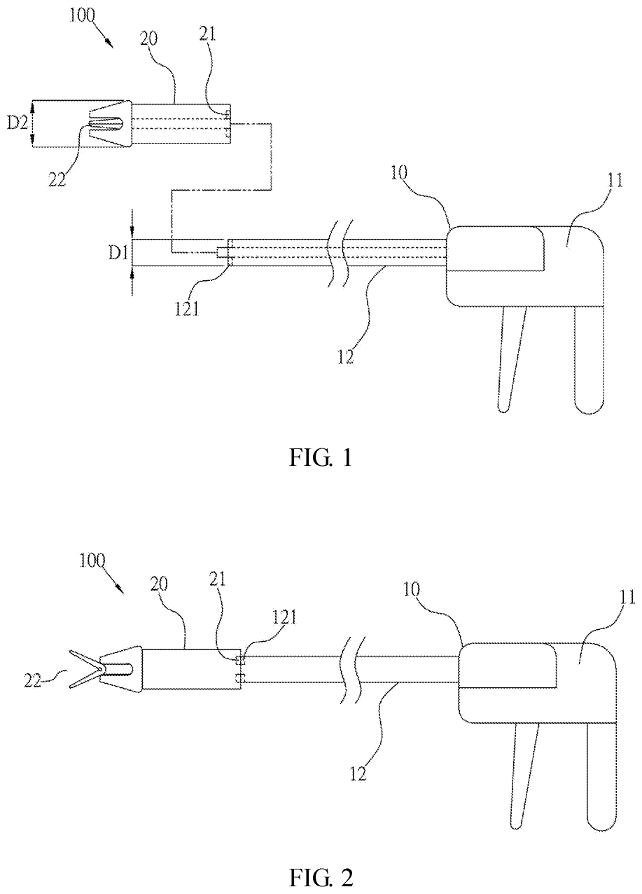

Referring to FIGS. 1-5, which respectively depict the first structure, the second structure, the structure of the hand piece, the structure of the hand piece and surgical piece, and the example of implementation of the intracorporeal assembled device for minimally invasive surgery of the present invention.

Referring to FIGS. 1 and 2, which depict the intracorporeal assembled device for minimally invasive surgery 100 for laparoscopic surgery according to an embodiment of the present invention. The intracorporeal assembled device for minimally invasive surgery 100 includes the hand piece 10 and the surgical piece 20. The hand piece 10 includes the handle portion 11, and the handle portion 11 has the pipe portion 12 disposed thereon. The pipe portion 12 has the first diameter D1, and the first connection portion 121 is disposed to one end of the pipe portion 12. The surgical piece 20 has the second diameter D2, and the second connection portion 21 corresponding to the first connection portion 121 is disposed to one end of the surgical piece 20. The workpiece 22 is disposed to one other end of the surgical piece 20. The first diameter D1 is smaller than the second diameter D2. The surgical piece 20 is connected to the pipe portion 12 in a detachable manner via the first connection portion 121 and the second connection portion 21. According to an embodiment of the present invention, the first connection portion and the second connection portion are a mortise-and-tenon structure.

In a better embodiment, the first diameter D1 is smaller than 3.5 mm or equal to 3.5 mm, while the second diameter D2 is greater than 3.5 mm. preferably, the second diameter D2 may be greater than 3.5 mm. In such configuration, the intracorporeal assembled device for minimally invasive surgery 100 may properly enter an opening of a wound equal to or smaller than 3.5 mm. Besides, the surgical piece 20 may ensures sufficient dimension for disposal of related members or suitable structures.

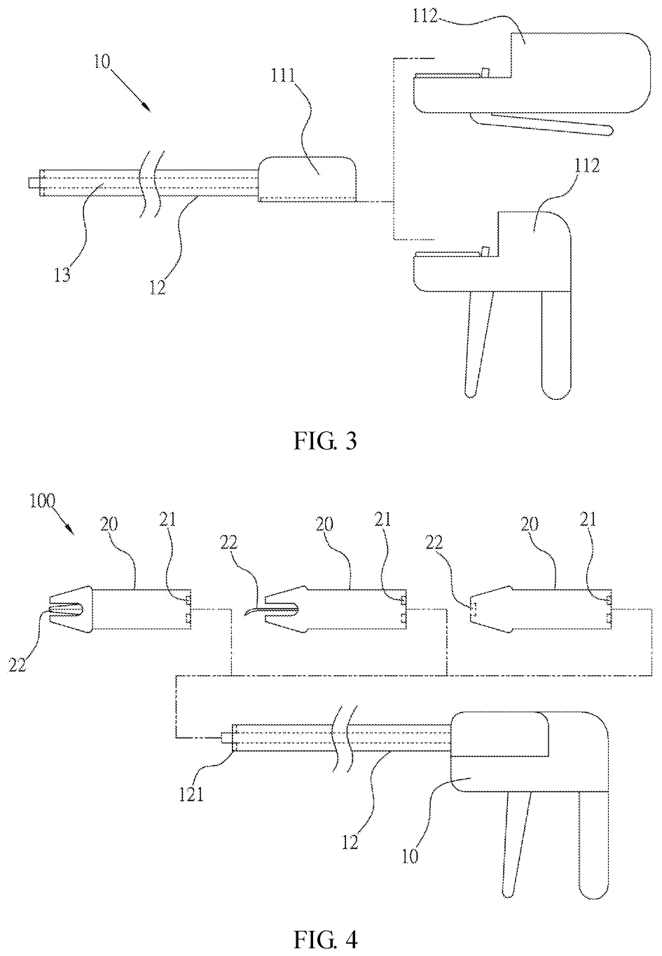

In addition, as shown in FIG. 3, the hand piece 10 may be a straight handle or a horizontal handle, and the handle portion 11 thereby includes the fixed handle portion 111 and the substitutive handle portion 112. In this way, the pipe portion 12 is disposed to the fixed handle portion 11 and the substitutive handle portion 112 is connected to the fixed handle portion 111 in a detachable manner. The substitutive handle portion 112 may be of various types such as the straight handle type or the horizontal handle type. Accordingly, the substitutive handle portion 112 may be substituted by the user to fit the requirement. For example, the straight handle is substituted for the horizontal handle, or the horizontal handle is substituted for the straight handle, which improves the convenience of use.

By the way, the fixed handle portion 111 and the substitutive handle portion 112 connect to each other in a detachable manner and the structure or means applied therebetween for the connection may be well known by a person skilled in the art. For example, the aforementioned structure may include slide rails in combination with slide blocks or may include grooves in combination with groove hooks. According to an embodiment of the present invention, the connection structure includes slide rails in combination with slide blocks. Redundant description is omitted. The substitutive handle portion 112 may include a linkage portion. The linkage portion may jam in a predetermined structure of the fixed handle portion 111, such as a groove, after the fixed handle portion 111 combines with the substitutive handle portion 112. In this way, the user may control the entire hand piece 10 and drive the workpiece 22 as well.

Furthermore, as shown in FIG. 4, the first connection portion 121 is disposed to one end of the pipe portion 12, and the second connection portion 21 corresponding to the first connection portion 121 is disposed to one end of the surgical piece 20. In this way, the surgical piece 20 may be connected to the pipe portion 12 in a detachable manner via the first connection portion 121 and the second connection portion 21. In a better embodiment, the first connection portion 121 and the second connection portion 21 may be magnetic members of different magnetic poles. Alternatively, the first connection portion 121 and the second connection portion 21 may be members combining with each other as a buckle set. The first connection portion 121 and the second connection portion 21 may be respectively magnetic members of different magnetic poles as an exemplary aspect in one embodiment of the present invention, while the first connection portion 121 and the second connection portion 21 may be members combining with each other as a buckle set in another embodiment. The aforementioned structure or means is well known by a person skilled in the art and the redundant description is hence omitted.

Accordingly, the surgical piece 20 may be substituted by the user to fit the requirement so as to improve the convenience of use. For example, the workpiece 22 may be a clamp, a stitching tool, a knotting tool or an energy device. Therefore, the user may substitute the surgical piece 20 having a knotting tool as the workpiece 22 for the surgical piece 20 having a clamp as the workpiece 22. In another way, the user may substitute the surgical piece 20 having a clamp as the workpiece 22 for the surgical piece 20 having a stitching tool as the workpiece 22.

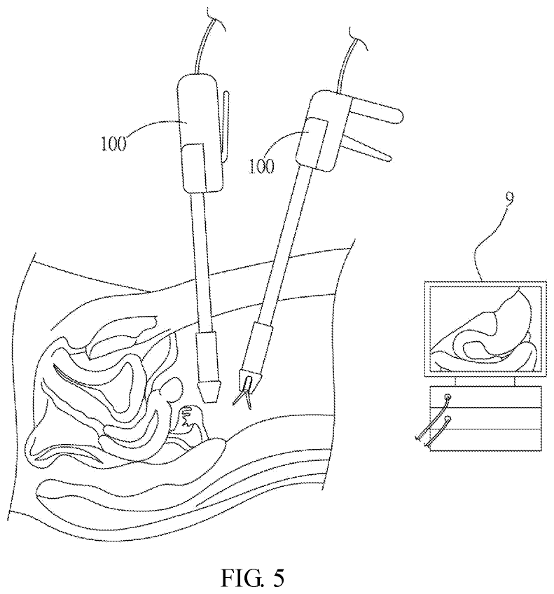

As shown in FIG. 5, in a better embodiment, the workpiece 22 is a camera. The hand piece 10 further includes a wire 13. The wire 13 connects to the handle portion 12. Further, the wire 13 connects to the surgical piece 20 so as to connect to the camera through the pipe portion 12. Accordingly, the intracorporeal assembled device for minimally invasive surgery 100 may use the camera to shoot instant images related to the surgery after the camera enters an opening of a wound equal to or smaller than 3.5 mm. The handle portion 11 may surely connect to the electronic device 9 to display the instant images or related messages.

It is noted that when the workpiece 22 is a clamp, a stitching tool or a knotting tool, the wire 13 may act as a transmission mechanism. In this way, the hand piece 10 may control the wire 13, which drives the workpiece 22 to perform some operation (e.g., grabbing and cutting) or to perform some even more complicated operation (e.g., stitching and knotting). The pipe portion 12 may surely include some structures (e.g., a pusher, bending driver having multi-degree of operation freedom and snake bone of ball joints) to aid the workpiece 22 to perform some operation (e.g., grabbing, dissection and cutting) or to perform some even more complicated operation (e.g., stitching and knotting).

Consequently, the intracorporeal assembled device for minimally invasive surgery provided in the present invention may be used in the mini-laparoscopic surgery for an opening of a wound equal to or smaller than 3.5 mm so as to enhance the applicability of the needle operation in the minimally invasive surgery.

The above description is merely illustrative and not restrictive. Any equivalent modification or change without departing from the spirit and scope of the present disclosure should be included in the appended claims.

What is claimed is:

1. An intracorporeal assembled device for minimally invasive surgery, applied to a laparoscopic surgery, the instrument comprises:
a hand piece, having a handle portion, the handle portion having a pipe portion, the pipe portion having a first diameter, and a first connection portion being disposed to one end of the pipe portion; and
a surgical piece, having a second diameter, a second connection portion corresponding to the first connection portion being disposed to one end of the surgical piece, and a workpiece being disposed to one other end of the surgical piece;
wherein the first diameter is smaller than the second diameter, and the surgical piece is connected to the pipe portion in a detachable manner via the first connection portion and the second connection portion.

2. The intracorporeal assembled device for minimally invasive surgery of claim 1, wherein the first diameter is smaller than or equal to 3.5 mm.

3. The intracorporeal assembled device for minimally invasive surgery of claim 2, wherein the second diameter is greater than 3.5 mm.

4. The intracorporeal assembled device for minimally invasive surgery of claim 1, wherein the first connection portion and the second connection portion are magnetic members of different magnetic poles.

5. The intracorporeal assembled device for minimally invasive surgery of claim 1, wherein the first connection portion and the second connection portion are members combining with each other as a buckle set.

6. The intracorporeal assembled device for minimally invasive surgery of claim 1, wherein the hand piece comprises a wire, the wire connects to the handle portion to be controlled by the handle portion, and the wire passes through the pipe portion and connects to the surgical piece.

7. The intracorporeal assembled device for minimally invasive surgery of claim 6, wherein the workpiece is a camera.

8. The intracorporeal assembled device for minimally invasive surgery of claim 1, wherein the hand piece is a straight handle or a horizontal handle.

9. The intracorporeal assembled device for minimally invasive surgery of claim 8, wherein the handle portion further comprises a fixed handle portion and a substitutive handle portion, the pipe portion is disposed to the fixed handle portion, and the substitutive handle portion connects to the fixed handle portion in a detachable manner.

10. The intracorporeal assembled device for minimally invasive surgery of claim 1, wherein the workpiece is a clamp, a stitching tool, a knotting tool or an energy device.

11. The intracorporeal assembled device for minimally invasive surgery of claim 1, wherein the first connection portion and the second connection portion are a mortise-and-tenon structure.
